(19) United States
(12) Patent Application Publication
(10) Pub. No.: US 2018/0088126 A1
(43) Pub. Date: Mar. 29, 2018

(54) METHOD OF IDENTIFYING PROTEINS IN HUMAN SERUM INDICATIVE OF PATHOLOGIES OF HUMAN LUNG TISSUES

(71) Applicant: Cancer, Prevention, and Cure, Ltd., Michigan City, IN (US)

(72) Inventors: Sung H. BAEK, Snohomish, WA (US); Robert T. Streeper, San Antonio, TX (US); Elzbieta Izbicka, San Antonio, TX (US)

(21) Appl. No.: 15/440,898

(22) Filed: Feb. 23, 2017

Related U.S. Application Data
(63) Continuation of application No. 14/465,549, filed on Aug. 21, 2014, now abandoned.
Application No. 14/465,549 is a continuation of application No. 12/208,876, filed on Sep. 11, 2008, now abandoned.
(60) Provisional application No. 60/971,422, filed on Sep. 11, 2007.

(51) Int. Cl. G01N 33/68; G01N 33/574

(52) U.S. Cl. CPC G01N 33/6848; G01N 33/6893; G01N 2500/00; G01N 2800/12; G01N 2800/122; G01N 33/57423

(57) ABSTRACT

A method of identifying proteins present in human serum which are differentially expressed between normal individuals and patients known to have non-small cell lung cancers and asthma, as diagnosed by a physician. Human serum specimens from each population are digested with trypsin or any other suitable endoproteinase and analyzed using a liquid chromatography electrospray ionization mass spectrometer. Mass spectral data from each population is compared to determine proteins with expression intensities which are significantly differentially expressed between the normal, asthma, and lung cancer populations. Eleven proteins are found to have expression intensities which are significantly differentially expressed between the populations. Finally, the identities of the eleven proteins are obtained by comparing the mass spectral data with known databases having libraries of mass spectral data of known proteins.

This is an original non-provisional application claiming benefit of U.S. Provisional Application 60/971,422 filed on Sep. 11, 2007, which is incorporated herein by reference.

### BACKGROUND OF THE INVENTION

### 1. Field of the Invention

The present invention relates generally to the diagnosis of pathologies of human lung tissues. More specifically, the present invention relates to the diagnosis of non-small cell lung cancers and asthma using liquid chromatography-mass spectrometry to identify proteins present in human sera which, when altered in terms of relative intensity of expression in the human serum from the same proteins found in a normal population, are indicative of pathologies associated with human lung tissues and the human respiratory system. By identifying the proteins associated with such pathologies, determining representative expression intensities, and comparing those expression intensities to the expression intensities present in the serum of a patient, it is possible to detect the presence of the pathologies early on in their progression through simple blood tests and to differentiate among the pathologies.

### 2. Description of the Related Art

Pathologies of the respiratory system, such as asthma and lung cancer, affect millions of Americans. In fact, the American Lung Association reports that almost 20 million Americans suffer from asthma. The American Cancer Society estimated 229,400 new cancer cases of the respiratory system and 164,840 deaths from cancers of the respiratory system in 2007 alone. While the five year survival rate of cancer cases when the cancer is detected while still localized is 46%, the five year survival rate of lung cancer patients is only 13%. Correspondingly, only 16% of lung cancers are discovered before the disease has spread. Lung cancers are generally categorized as two main types based on the pathology of the cancer cells. Each type is named for the types of cells that were transformed to become cancerous. Small cell lung cancers are derived from small cells in the human lung tissues, whereas non-small-cell lung cancers generally encompass all lung cancers that are not small-cell type. Non-small cell lung cancers are grouped together because the treatment is generally the same for all non-small-cell types. Together, non-small-cell lung cancers, or NSCLCs, make up about 75% of all lung cancers.

A major factor in the diminishing survival rate of lung cancer patients is the fact that lung cancer is difficult to diagnose early. Current methods of diagnosing lung cancer or identifying its existence in a human are restricted to taking X-rays, CT scans and similar tests of the lungs to physically determine the presence or absence of a tumor. Therefore, the diagnosis of lung cancer is often made only in response to symptoms which have presented for a significant period of time, and after the disease has been present in the human long enough to produce a physically detectable mass.

Similarly, current methods of detecting asthma are typically performed long after the presentation of symptoms such as recurrent wheezing, coughing, and chest tightness. Current methods of detecting asthma are typically restricted to lung function tests such as spirometry tests or challenge tests. Moreover, these tests are often ordered by the physician to be performed along with a multitude of other tests to rule out other pathologies or diseases such as chronic obstructive pulmonary disease (COPD), bronchitis, pneumonia, and congestive heart failure.

There does not exist in the prior art a simple, reliable method of diagnosing pathologies of human lung tissues early in their development. Furthermore, there is not a blood test available today which is capable of indicating the presence of a particular lung tissue pathology. It is therefore desirable to develop a method to determine the existence of lung cancers early in the disease progression. It is likewise desirable to develop a method to diagnose asthma and non-small cell lung cancer and to differentiate them from each other and from other lung diseases such as infections at the earliest appearance of symptoms. It is further desirable to identify specific proteins present in human blood which, when altered in terms of relative intensities of expression, are indicative of the presence of non-small cell lung cancers and/or asthma.

### BRIEF SUMMARY OF THE INVENTION

The present invention provides a novel method of identifying proteins present in human serum which are differentially expressed between normal individuals and patients known to have non-small cell lung cancers and asthma, as diagnosed by a physician, using a liquid chromatography electrospray ionization mass spectrometer (“LC-ESIMS”). Selection of proteins indicative of non-small cell lung cancers and/or asthma was made by comparing the mass spectral data, namely the mass of peptides and graphical indications of the intensities of the proteins expressed across time in a single dimension. Thousands of proteins were compared, resulting in the selection of eleven proteins which were expressed in substantially differing intensities between populations of individuals not having any lung tissue pathologies, populations of individuals having asthma, as diagnosed by a physician, and populations of individuals having non-small cell lung cancers, as diagnosed by a physician.

Specifically, human sera were obtained from a “normal population,” an “asthma population”, and a “lung cancer population.” “Normal population,” as used herein is meant to define those individuals known not to have asthma or lung cancers. “Asthma population,” as used herein, is meant to define those individuals which were known to have asthma and diagnosed as such by a physician. “Lung cancer population,” as used herein, is meant to define those individuals which were known to have non-small cell lung cancers and diagnosed as such by a physician.

After obtaining the sera of the normal population, asthma population and lung cancer population, each serum specimen was divided into aliquots and exposed to a digesting agent or protease, namely, trypsin, to digest the proteins present in the serum specimens into defined and predictable cleavages or peptides. The peptides created by the enzymatic action of trypsin, commonly known as the tryptic peptides, were then separated from the insoluble matter digested by the trypsin by subjecting the specimens to a centrifugation to precipitate insoluble matter. The supernatant solution containing the tryptic peptides was then subjected to capillary liquid chromatography to effect tempero-spatial separation of the tryptic peptides.

The tryptic peptides were then subjected to an LC-ESIMS. Each peptide was separated in time by passing the peptide through a column of hydrophobic fluid, namely, water, acetonitrile containing 0.1% by volume formic acid over a chromatographic column containing Supelcosil ABZ+ 5 μm packing material stationary phase with a bed length of 18 cm and an internal diameter of 0.375 mm. The separated peptides are carried by a column effluent. The column has a terminus from which the separated peptides were then electrosprayed by application of a high voltage to the column tip having a positive bias relative to ground, forming a beam of charged droplets that were accelerated toward the inlet of the LC-ESIMS by the force of the applied electrical field. The resulting spray formed consisted of small droplets of solvent containing dissolved tryptic peptides. The droplets were desolvated by passage across an atmospheric pressure region of the electrospray source and then into a heated capillary inlet of the LC-ESIMS.

The desolvation of the droplets resulted in the deposition of positively charged ions, most typically hydrogen (H⁺) on the peptides, imparting charge to the peptides. Such charged peptides in the gas phase are described in the art as “pseudo-molecular ions.” The pseudo-molecular ions are drawn through various electrical potentials into a mass analyzer of the LC-ESIMS, wherein they are separated in space and time on the basis of the mass to charge ratio. Once separated by mass to charge ratio, the pseudo-molecular ions are then directed by additional electric field gradients into a detector of the LC-ESIMS, wherein the pseudo-molecular ion beam is converted into electrical impulses that are recorded by data recording devices.

Thus, the peptides present in the tryptic digest were passed to the mass analyzer in the LC-ESIMS where molecular weights were measured for each peptide, producing time incremented mass spectra that are acquired repeatedly over the entirety of the time that the peptides from the sample are passing out of the column. The mass spectral readouts are generally graphic illustrations of the peptides found by the LC-ESIMS, wherein the x-axis is the measurement mass to charge ratio, the y-axis is the signal intensity of the peptide. These mass spectra can then be assembled in time into a three dimensional display wherein the x-axis is the time of the chromatographic separation, the z-axis is the mass axis of the mass spectrum and the y-axis is the intensity of the mass spectral signals, which is proportional to the quantity of a given pseudo-molecular ion detected by the LC-ESIMS.

Next, comparative analysis was performed comparing the mass spectral readouts for each specimen tested from the asthma population and the lung cancer population to each specimen tested from the normal population. Each tryptic peptide pseudo-molecular ion signal (“peak”) associated with a putatively identified protein that was detected in the LC-ESIMS was compared across asthma, lung cancer and normal pathologies. Peptides with mass spectral peak intensities that indicated the peptide quantities were not substantially altered when comparing the asthma population or lung cancer population to the normal population were determined to be insignificant and excluded. Generally, the exclusion criteria used involved comparing the peptide peak intensities for at least half of the identified characteristic peptides for a given protein across at least ten data sets derived from the analysis of individual patient sera from each pathology. If the intensity of the majority of peptide peaks derived from given protein were at least 10 fold higher in intensity for 80% of the serum data sets, the protein was classed as differentially regulated between the two pathologic classes.

As a result of the comparative analysis, eleven proteins were determined to be consistently differentially expressed between the asthma population, lung cancer population and normal population. The eleven proteins were identified by reference to known databases or libraries of proteins and peptides. Examples of such databases include Entrez Protein maintained by the National Center for Biotechnology Information “NCBInr”), ExPASy maintained by the Swiss Bioinformatics Institute (“SwissProt”), and the Mass Spectral Database (“MSDB”) of the Medical Research Council Clinical Science Center of the Imperial College of London.

The mass spectral readouts for each specimen from each of the normal, lung cancer and asthma population were inputted into a known search engine called Mascot. Mascot is a search engine known in the art which uses mass spectrometry data to identify proteins from four major sequencing databases, namely the MSDB, NCBInr, SwissProt and dbEST databases. Search criteria and parameters were inputted into the Mascot program and each specimen was run through the Mascot program. The Mascot program then ran the peptides inputted against the sequencing databases, comparing the peak intensities and masses of each peptide to the masses and peak intensities of known peptides and proteins. Mascot then produced a candidate list of possible matches, commonly known as “significant matches” for each peptide that was run.

Significant matches are determined by the Mascot program by assigning a score called a “Mowse score” for each specimen tested. The Mowse score is an algorithm wherein the score is −10*LOG₁₀(P), where P is the probability that the observed match is a random event, which correlates into a significance p value where p is less than 0.05, which is the generally accepted standard of significance in the scientific community. Mowse scores of approximately 55 to approximately 66 or greater are generally considered significant. The significance level varies somewhat due to specific search considerations and database parameters. The significant matches were returned for each peptide run, resulting in a candidate list of proteins.

The peptides were then matched to the proteins from the significant matches to determine the identity of the peptides run through the Mascot program. Manual analysis was performed for each peptide identified by the Mascot program and each protein from the significant matches. The peak intensity matches which were determined to be the result of “noise”, whether chemical or electronic were excluded. The data from the mass spectral readouts were cross checked with the significant matches to confirm the raw data, peak identities, charge multiplicities, isotope distribution and flanking charge states.

A reverse search was then performed to add peptides to the candidate list which may have been missed by the automated search through the Mascot program. The additional peptides were identified by selecting the “best match” meaning the single protein which substantially matched each parameter of the peptide compared, performing an in silico digest wherein the tryptic peptides and their respective molecular masses are calculated based on the known amino acid or gene sequence of the protein. These predicted peptide masses are then searched against the raw mass spectral data and any peaks identified are examined and qualified as described above. Then, all of the peptides including those automatically identified by Mascot and those identified by manual examination are entered into the mass list used by Mascot. The refined match is then used to derive the refined Mowse score, as discussed herein below.

As a result of the identification process, the eleven proteins determined to be significantly differentially expressed between the asthma population, lung cancer population and/or normal population were identified as BAC04615, Q6NSC8, CAF17350, Q6ZUD4, Q8N7P1, CAC69571, FERM domain containing protein 4, JC1445 proteasome endopetidase complex chain C2 long splice, Syntaxin 11, AAK13083, and AAK130490. BAC04615, Q6NSC8, CAF 17350, Q6ZUD4, Q8N7P1 are identified proteins resulting from genetic sequencing efforts. FERM domain containing protein 4 is known to be involved in intracytoplasmic protein membrane anchorage. JC1445 proteasome endopetidase complex chain C2 long splice is a known proteasome. Syntaxin 11 is active in cellular immune response. BAC04615, AAK13083, and AAK130490 are major histocompatibility complex (“MHC”) associated proteins.

### BRIEF DESCRIPTION OF THE DRAWINGS

FIG. 1 discloses a table showing Mowse scores and significant matches for the protein BAC04615;

FIG. 2 discloses a table showing Mowse scores and significant matches for the protein Q6NSC8;

FIG. 3 discloses a table showing Mowse scores and significant matches for the protein CAF17350;

FIG. 4 discloses a table showing Mowse scores and significant matches for the protein Q6ZUD4;

FIG. 5 discloses a table showing Mowse scores and significant matches for the protein Q8N7P1;

FIG. 6 discloses a table showing Mowse scores and significant matches for the protein CAC69571;

FIG. 7 discloses a table showing Mowse scores and significant matches for the protein FERM 4 domain containing protein 4;

FIG. 8 discloses a table showing Mowse scores and significant matches for the protein JC1445 proteasome endopetidase complex chain C2 long splice;

FIG. 9 discloses a table showing Mowse scores and significant matches for the protein Syntaxin 11;

FIG. 10 discloses a table showing Mowse scores and significant matches for the proteins AAK13083 and AAK13049.

### DETAILED DESCRIPTION OF THE INVENTION

The present invention provides a method of identifying, and identifies proteins present in human serum which are differentially expressed between normal individuals and patients known to have non-small cell lung cancers and asthma, as diagnosed by a physician, using liquid chromatography electrospray ionization mass spectrometry. By determining the proteins which are substantially and consistently differentially expressed between populations of people not having any pathologies of human lung tissues, populations of people diagnosed with asthma, and populations of people diagnosed with non-small cell lung cancers, and obtaining the identity of those proteins, it is possible to identify the presence of the pathology in a patient through blood tests identifying the same proteins and quantifying the expression levels of the proteins to identify and diagnose asthma or non-small cell lung cancer much earlier in the progression of the respective diseases.

Human blood samples were collected from volunteers. Thirty samples were collected from individuals known not to have either non-small cell lung cancer or asthma. The individuals known not to have either non-small cell lung cancer or asthma comprise, and are referred to herein as, the “normal population.” Furthermore, the term “lung cancer”, as used herein, is meant to describe non-small cell lung cancers. Twenty-eight blood samples were collected from individuals known to have asthma and diagnosed as such by a physician. The individuals known to have asthma comprise, and are referred to herein as, the “asthma population.” Thirty blood samples were collected from individuals known to have non-small cell lung cancers and diagnosed as such by a physician. The individuals known to have non-small cell lung cancer comprise, and are referred to herein as the “lung cancer population.” Generally, as used herein, the term “lung cancer” or “lung cancers” is meant to refer to non-small cell lung cancers. Finally, seventy-one blood samples were collected from individuals known to have risks of lung cancer due to a history of cigarette smoking as recorded by a physician. These seventy one samples are the subject of ongoing research and experimentation, and are accordingly not discussed herein.

The blood samples were collected from volunteers under an IRB approved protocol, following informed consent using standard venipuncture techniques into sterile 10 ml BD Vacutainer® glass serum red top tubes. The blood samples were then left undisturbed at room temperature for thirty minutes to allow the blood to clot. The samples were spun in a standard benchtop centrifuge at room temperature at two thousand rpm for ten minutes to separate the serum from the blood samples. The serum of each sample was then removed by pipetting the serum into secondary tubes. The secondary tubes were pre-chilled on ice to ensure the integrity of each serum specimen by limiting degradation due to proteolysis and denaturation. The serum specimens from each sample collected were then divided into 1.0 ml aliquots in pre-chilled Cryovial tubes on ice. The aliquots from the serum specimens were stored at a temperature at least as cold as eighty degrees below Celsius (−80° C.). The processing time was no more than one hour from phlebotomy to storing at −80° C.

Eight to ten serum specimens from each of the asthma population, normal population and lung cancer population were selected at random to be tested. Each serum specimen from each population was subjected to a protease or digesting agent, in this case, trypsin. Trypsin was used as the protease, and is desirable to be used as a protease because of its ability to make highly specific and highly predictable cleavages due to the fact that trypsin is known to cleave peptide chains at the carboxyl side of the lysine and arginine, except where a proline is present immediately following either the lysine or arginine. Although trypsin was used, it is possible to use other proteases or digesting agents. It is desirable to use a protease, or mixture of proteases, which cleave at least as specifically as trypsin.

The tryptic peptides, which are the peptides left by the trypsin after cleavage, were then separated from the insoluble matter by subjecting the specimens to a centrifugation and a capillary liquid chromatography, with an aqueous acetonitrile gradient with 0.1% formic acid using a 0.375×180 mm Supelcosil ABZ+ column on an Eksigent 2D capillary HPLC to effect chromatographic resolution of the generated tryptic peptides. This separation of the peptides is necessary because the electrospray ionization process is subject to ion co-suppression, wherein ions of a type having a higher proton affinity will suppress ion formation of ions having lower proton affinities if they are simultaneously eluting from the electrospray emitter, which in this case is co-terminal with the end of the HPLC column.

This methodology allows for the separation of the large number of peptides produced in the tryptic digestions and helps to minimize co-suppression problems, thereby maximizing chances of the formation of pseudo-molecular ion co-suppression, thereby maximizing ion sampling. The tryptic peptides for each specimen were then subjected to an LC-ESIMS. The LC-ESIMS separated each peptide in each specimen in time by passing the peptides in each specimen through a column of solvent system consisting of water, acetonitrile and formic acid as described above.

The peptides were then sprayed with in an electrospray ionization source to ionize the peptides and produce the peptide pseudo-molecular ions as described above. The peptides were passed through a mass analyzer in the LC-ESIMS where molecular masses were measured for each peptide pseudo-molecular ion. After passing through the LC-ESIMS, mass spectral readouts were produced for the peptides present in each sample from the mass spectral data, namely the intensities the molecular weights and the time of elution from a chromatographic column of the peptides. The mass spectral readouts are generally graphic illustrations of the peptide pseudo-molecular ion signals recorded by the LC-ESIMS, wherein the x-axis is the measurement of mass to charge ratio, the y-axis is the intensity of the pseudo-molecular ion signal. These data are then processed by a software system that controls the LC-ESIMS and acquires and stores the resultant data.

Once the mass spectral data was obtained and placed on the mass spectral readouts, a comparative analysis was performed wherein the mass spectral readouts of each serum specimen tested in the LC-ESIMS for each population was performed, both interpathologically and intrapathalogically. The mass spectral peaks were compared between each specimen tested in the normal population. The mass spectral peaks were then compared between each specimen tested in the asthma population and the lung cancer population. Once the intrapathological comparisons were performed, interpathological comparisons were performed wherein the mass spectral readouts for each specimen tested in the LC-ESIMS for the asthma population was compared against each specimen tested in the normal population. Likewise, the mass spectral readouts for each specimen tested in the LC-ESIMS for the lung cancer population was compared against each specimen tested in the normal population.

Peptides with mass spectral readouts that indicated the peptide intensities were inconsistently differentially expressed intrapathologically or were not substantially altered (less than 10 fold variance in intensity) when comparing the asthma population or lung cancer population to the normal population were determined to be insignificant and excluded. Generally, the exclusion criteria used involved comparing the peptide peak intensities for at least half of the identified characteristic peptides for a given protein across at least ten data sets derived from the analysis of individual patient sera from each pathology. If the intensity of the majority of peptide peaks derived from given protein were at least 10 fold higher in intensity for 80% of the serum data sets, the protein was classed as differentially regulated between the two pathologic classes.

However, the identity of the proteins giving rise to the peptides that were observed to be differentially regulated were unknown and needed to be identified. To make the identification of the proteins, peptide pseudo-molecular ion signal intensities were compared across known databases which contain libraries of known proteins and peptides and suspected proteins and peptides.

The mass spectral readouts of the tryptic digests for each specimen from each of the normal, lung cancer and asthma population were inputted into a known search engine called Mascot. Mascot is a search engine known in the art which uses mass spectrometry data to identify proteins from four major sequencing databases, namely the MSDB, NCBInr, SwissProt and dbEST databases. These databases contain information on all proteins of known sequence and all putative proteins based on observation of characteristic protein transcription initiation regions derived from gene sequences. These databases are continually checked for accuracy and redundancy and are subject to continuous addition as new protein and gene sequences are identified and published in the scientific and patent literature.

As a result of the comparative analysis, eleven proteins were determined to be consistently differentially expressed between the asthma population, lung cancer population and normal population. Search criteria and parameters were inputted into the Mascot program and the mass spectral data from the mass spectral readouts for each population were run through the Mascot program. The mass spectral data entered into the Mascot program were for the all specimens of each pathology. The Mascot program then ran the mass spectral data for the peptides inputted against the sequencing databases, comparing the peak intensities and masses of each peptide to the masses and peak intensities of known peptides and proteins. Mascot then produced a search result which returned a candidate list of possible protein identification matches, commonly known as “significant matches” for each sample that was analyzed.

Significant matches are determined by the Mascot program by assigning a score called a “Mowse score” for each specimen tested. The Mowse score is an algorithm wherein the score is −10*LOG₁₀(P), where P is the probability that the observed match is a random event, which correlates into a significance p value where p is less than 0.05, which is the generally accepted standard in the scientific community. Mowse scores of approximately 55 to approximately 66 or greater are generally considered significant. The significance level varies somewhat due to specific search considerations and database parameters. The significant matches were returned for each peptide run, resulting in a candidate list of proteins.

Next, comparative analysis was performed comparing the mass spectral readouts for each specimen tested from the asthma population and the lung cancer population to each specimen tested from the normal population. Each tryptic peptide pseudo-molecular ion signal (peak) associated with an putatively identified protein that was detected in the LC-ESIMS was compared across asthma, lung cancer and normal pathologies. Peptides with mass spectral peak intensities that indicated the peptide quantities were not substantially altered when comparing the asthma population or lung cancer population to the normal population were determined to be insignificant and excluded. Generally, the exclusion criteria used involved comparing the peptide peak intensities for at least half of the identified characteristic peptides for a given protein across at least ten data sets derived from the analysis of individual patient sera from each pathology. If the intensity of the majority of peptide peaks derived from given protein were at least 10 fold higher in intensity for 80% of the serum data sets, the protein was classed as differentially regulated between the two pathologic classes.

The data from the mass spectral readouts were cross checked with the significant matches to confirm the raw data, peak identities, charge multiplicities, isotope distribution and flanking charge states. A reverse search was then performed to add peptides to the candidate list which may have been missed by the automated search through the Mascot program. The additional peptides were identified by selecting the “best match” meaning the single protein which substantially matched each parameter of the peptide compared, performing an in silico digest wherein the tryptic peptides and their respective molecular masses are calculated based on the known amino acid or gene sequence of the protein. These predicted peptide masses are then searched against the raw mass spectral data and any peaks identified are examined and qualified as described above. Then, all of the peptides including those automatically identified by Mascot and those identified by manual examination are entered into the mass list used by Mascot. The refined match is then used to derive the refined Mowse score, as presented below.

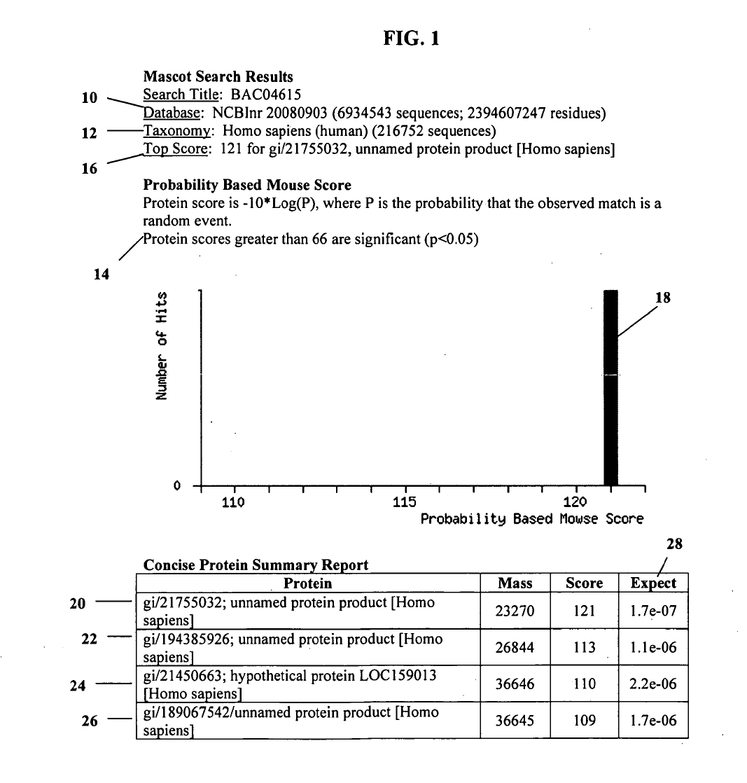

Referring to FIG. 1 through FIG. 10, Mascot search results are shown for each protein identified as differentially expressed between either the lung cancer population or the asthma population compared to the normal population. In each case, the search criteria and parameters were entered, and a Mowse score threshold for acceptability of significance was established. Referring to FIG. 1, a Mascot search result for the protein BAC04615 is shown. The database selected to be searched was NCBInr 10, and the taxonomy of the specimens entered into the Mascot program was set as Homo sapiens 12. The Mowse score threshold of significance was established as the Mowse value of sixty six or greater 14. As a result of the Mascot search, a top score of 121 was obtained, as indicated by Mowse score graph 18 the y-axis of the graph indicates the number of proteins identified having a particular Mowse score.

Still referring to FIG. 1, the top Mowse score of one hundred twenty one was given for gi/21755032, as indicated by row 20. A Mowse score of 121 is highly significant, meaning that there is a very low probability that the match is random. In fact, as indicated in column 28, the expectation that this match would occur at random is indicated by the Mascot program as 1.7×10⁻⁰⁷. However, the proteins indicated in rows 22, 24 and 26 also had very high Mowse scores, indicating that these three proteins are significant matches as well. The manual analysis was then performed, wherein insignificant and/or noise data was removed, and raw data, peak identities, charge multiplicities, isotope distribution and flanking charge states were cross checked. As a result of the manual analysis, the probability that the proteins indicated in rows 22, 24 and 26 are significant matches was significantly reduced, and thus, proteins indicated in rows 22, 24 and 26 were excluded as matches. The protein indicated in row 20, gi/21755032, was identified as the protein indicated by the mass spectral data entered into the Mascot program in FIG. 1. The protein number indicated in row 20, gi/21755032, where gi number (sometimes written as “GI”) is simply a series of digits that are assigned consecutively to each sequence record processed by NCBI. gi/21755032 corresponds to the protein BAC04615.

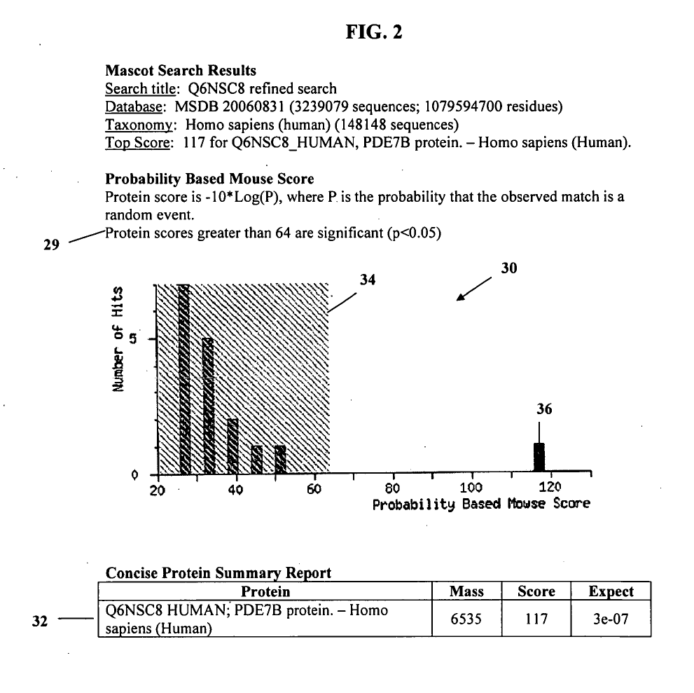

Referring to FIG. 2, a Mascot search result for the protein Q6NSC8 is disclosed. The Mowse score threshold of significance 29 was established as the Mowse value of sixty four, and a top Mowse score of one hundred seventeen was obtained, as indicated by Mowse score bar 36 in Mowse score graph 30. The protein identified which correlated to Mowse score bar 36 is Q6NSC8, as indicated in row 32. As shown in FIG. 2, the shaded portion 34 of the Mowse score graph 30 indicates proteins which were recorded, but which were below the threshold of significance, and thus, were eliminated from consideration.

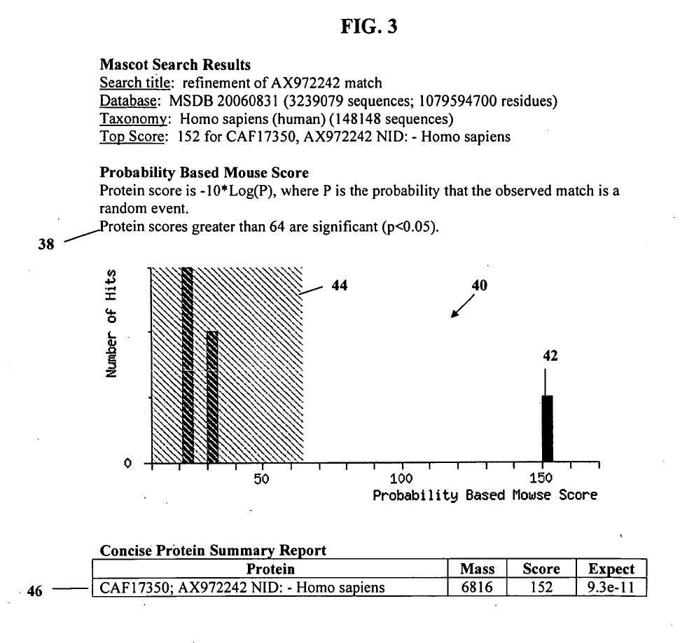

Referring to FIG. 3, a Mascot search result for the protein CAF17350 is disclosed. The Mowse score threshold of significance 38 was established as the Mowse value of sixty four, and a top Mowse score of one hundred fifty two was obtained, as indicated by Mowse score bar 42 in Mowse score graph 40. The protein identified which correlated to Mowse score bar 42 is CAF17350, as indicated in row 46. As shown in FIG. 3, the shaded portion 44 of the Mowse score graph 40 indicates proteins which were recorded, but which were below the threshold of significance, and thus, were eliminated from consideration.

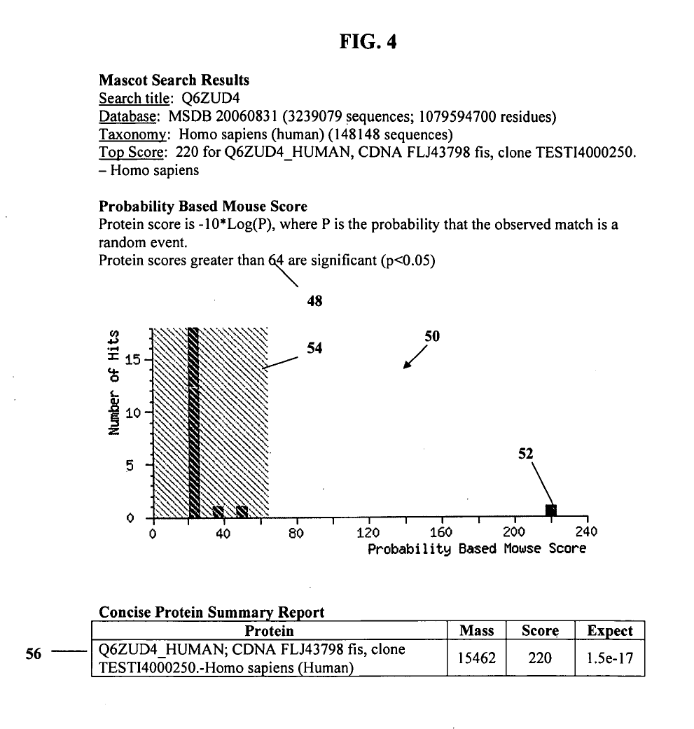

Referring to FIG. 4, a Mascot search result for the protein Q6ZUD4 is disclosed. The Mowse score threshold of significance 48 was established as the Mowse value of sixty four, and a top Mowse score of two hundred twenty was obtained, as indicated by Mowse score bar 52 in Mowse score graph 50. The protein identified which correlated to Mowse score bar 52 is Q6ZUD4, as indicated in row 56. As shown in FIG. 4, the shaded portion 54 of the Mowse score graph 50 indicates proteins which were recorded, but which were below the threshold of significance, and thus, were eliminated from consideration.

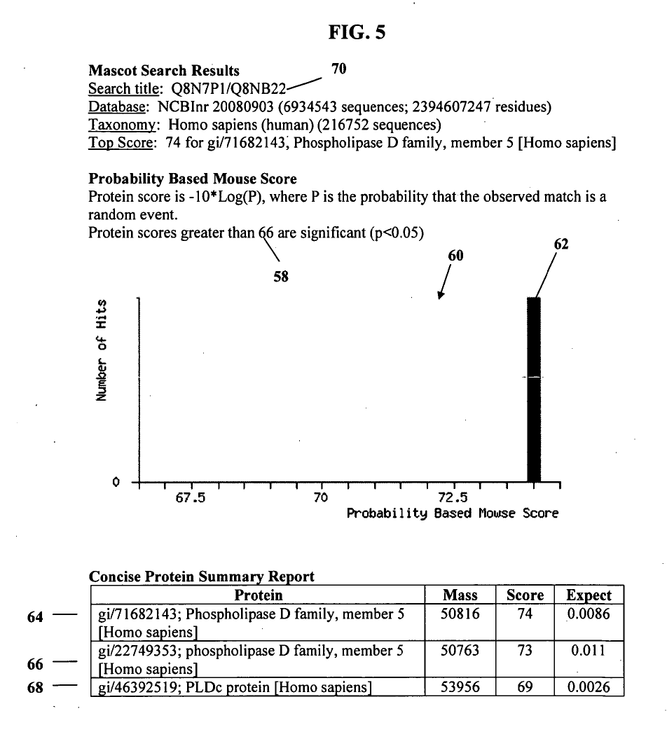

Referring to FIG. 5, a Mascot search result for the protein Q8N7P1 is disclosed. The Mowse score threshold of significance 58 was established as the Mowse value of sixty six, and a top Mowse score of seventy four was obtained, as indicated by Mowse score bar 62 in Mowse score graph 60. The protein identified which correlated to Mowse score bar 62 is gi/71682143, as indicated in row 64. Similarly to FIG. 1, gi/71682143 corresponds to protein Q8N7PI. The proteins indicated in rows 66 and 68 also had very high Mowse scores, indicating that these two proteins are significant matches as well. The manual analysis was then performed, wherein insignificant and/or noise data was removed, and raw data, peak identities, charge multiplicities, isotope distribution and flanking charge states were cross checked. As a result of the manual analysis, the probability that the proteins indicated in rows 66 and 68 are significant matches was significantly reduced, and thus, proteins indicated in rows 66 and 68 were excluded as matches. Q8N7PI was identified as the protein indicated by the mass spectral data entered into the Mascot program in FIG. 5. The indication at 70 to the protein Q8NB22 is indicated because it is the same protein as Q8N7PI.

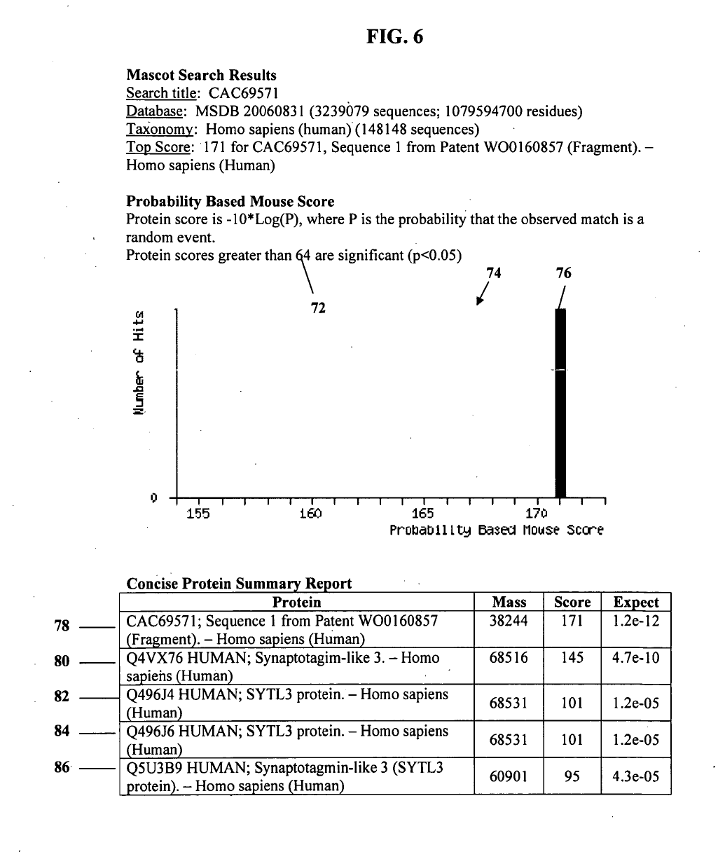

Referring to FIG. 6, a Mascot search result for the protein CAC69571 is disclosed. The Mowse score threshold of significance 72 was established as the Mowse value of sixty four, and a top Mowse score of one hundred seventy one was obtained, as indicated by Mowse score bar 76 in Mowse score graph 74. The protein indicated which correlated to Mowse score bar 76 is CAC69571, as indicated in row 78. The proteins indicated in rows 80, 82, 84 and 86 also had very high Mowse scores, indicating that these four proteins are significant matches as well. The manual analysis was then performed, wherein insignificant and/or noise data was removed, and raw data, peak identities, charge multiplicities, isotope distribution and flanking charge states were cross checked. As a result of the manual analysis, the probability that the proteins indicated in rows 80, 82, 84 and 86 are significant matches was significantly reduced, and thus, proteins indicated in rows 80, 82, 84 and 86 were excluded as matches. CAC69571 was identified as the protein indicated by the mass spectral data entered into the Mascot program in FIG. 6.

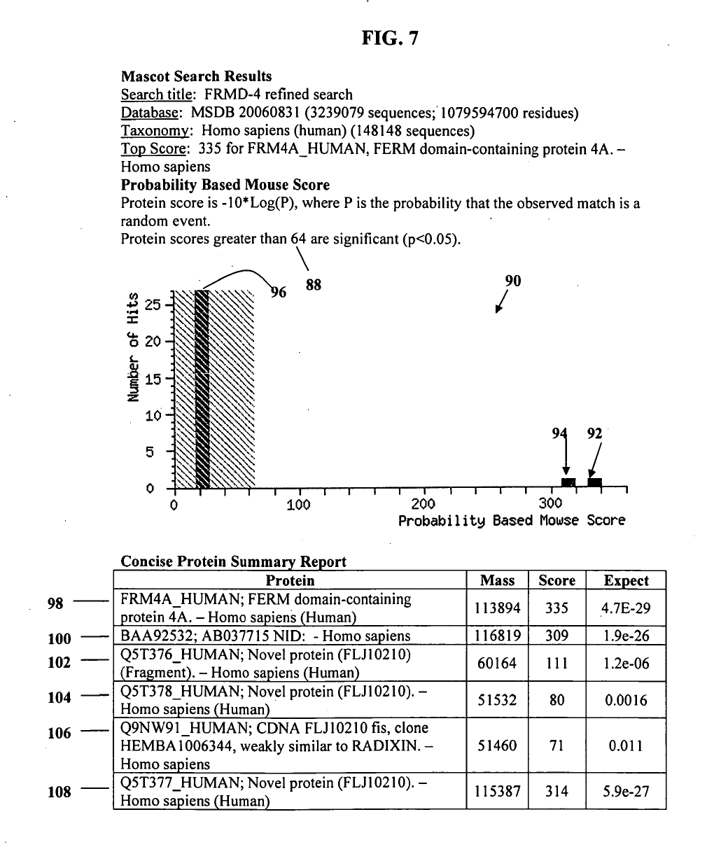

Referring to FIG. 7, a Mascot search result for the protein FERM 4 domain containing protein 4 is disclosed. The Mowse score threshold of significance 88 was established as the Mowse value of sixty four, and a top Mowse score of three hundred thirty five was obtained, as indicated by Mowse score bar 92 in Mowse score graph 90. The protein indicated which correlated to Mowse score bar 92 is FERM 4 domain containing protein 4, as indicated in row 98. The proteins indicated in rows 100, 102, 104 and 106 and 108 also had very high Mowse scores, indicating that these five proteins are significant matches as well. The manual analysis was then performed, wherein insignificant and/or noise data was removed, and raw data, peak identities, charge multiplicities, isotope distribution and flanking charge states were cross checked. As a result of the manual analysis, the probability that the proteins indicated in rows 100, 102, 104 and 106 and 108 are significant matches was significantly reduced, and thus, proteins indicated in rows 100, 102, 104 and 106 and 108 were excluded as matches. FERM 4 domain containing protein 4 was identified as the protein indicated by the mass spectral data entered into the Mascot program in FIG. 7.

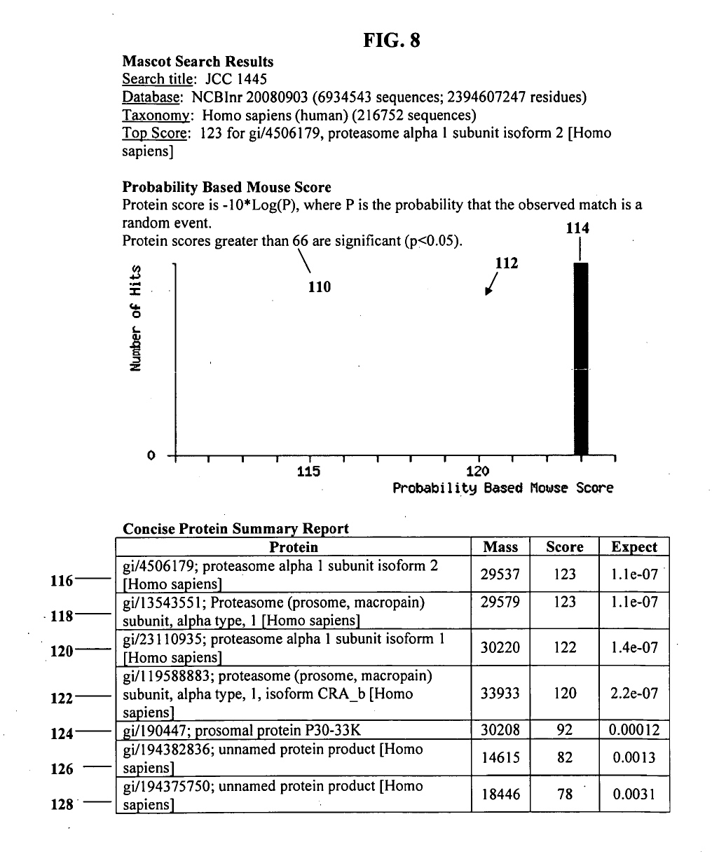

Referring to FIG. 8, a Mascot search result for the protein JCC1445 proteasome endopeptidase complex chain C2 long splice form (“JCC1445”) is disclosed. The Mowse score threshold of significance 110 was established as the Mowse value of sixty six, and a top Mowse score of one hundred twenty three was obtained, as indicated by Mowse score bar 114 in Mowse score graph 112. The protein identified which correlated to Mowse score bar 114 is gi/4506179, as indicated in row 116. gi/4506179 corresponds to protein JCC1445. The proteins indicated in rows 118, 120, 122, 124, 126 and 128 also had very high Mowse scores, indicating that these six proteins are significant matches as well. The manual analysis was then performed, wherein insignificant and/or noise data was removed, and raw data, peak identities, charge multiplicities, isotope distribution and flanking charge states were cross checked. As a result of the manual analysis, the probability that the proteins indicated in rows 118, 120, 122, 124, 126 and 128 are significant matches was significantly reduced, and thus, proteins indicated in rows 118, 120, 122, 124, 126 and 128 were excluded as matches. JCC1445 was identified as the protein indicated by the mass spectral data entered into the Mascot program in FIG. 8.

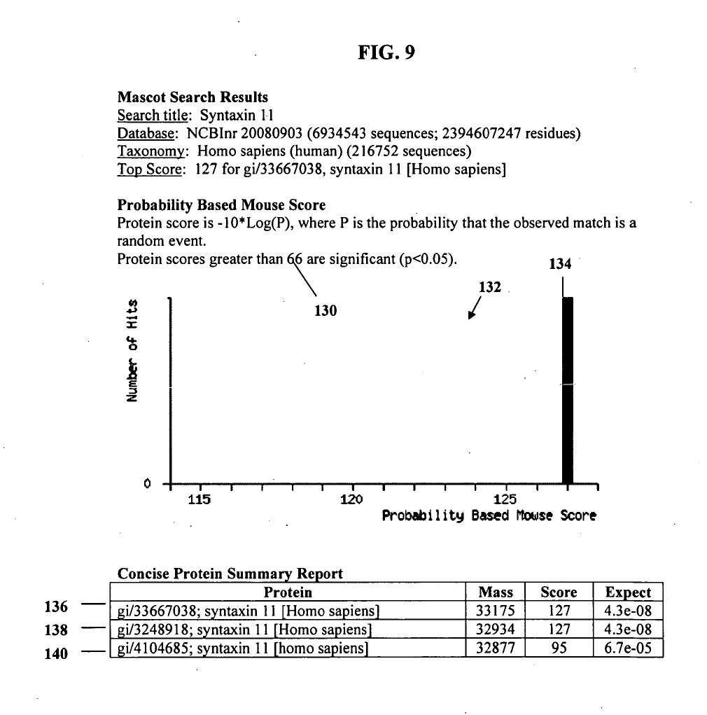

Referring to FIG. 9, a Mascot search result for the protein Syntaxin 11 is disclosed. The Mowse score threshold of significance 130 was established as the Mowse value of sixty six, and a top Mowse score of one hundred twenty seven was obtained twice, as indicated by Mowse score bars 134, and rows 136 and 138. A third Mowse score of 95 was obtained for Syntaxin 11, as indicated in row 140. Syntaxin 11 was identified as the protein indicated by the mass spectral data entered into the Mascot program in FIG. 9.

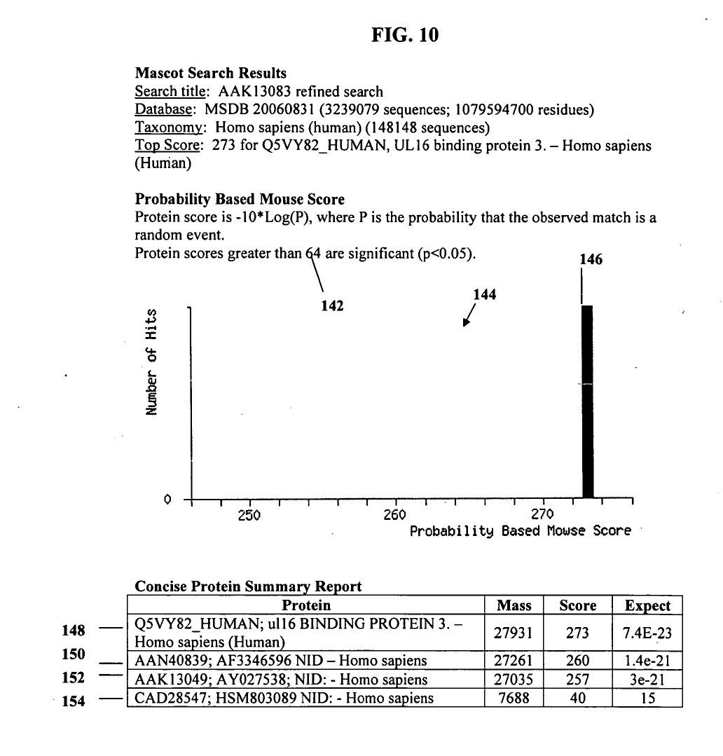

Referring to FIG. 10, Mascot search results for two proteins, AAK13083 and AAK13049 are disclosed. The Mowse score threshold of significance 142 was established as the Mowse value of sixty four, and a top Mowse score of two hundred seventy three was obtained by protein Q5VY82, as indicated in row 148 and Mowse score bar 146. The proteins indicated in rows 150, 152 and 154 also had very high Mowse scores, indicating that these three proteins are significant matches as well. However, as a result of the manual analysis performed, the proteins indicated in rows 150 and 154 were eliminated as probable matches. Q5VY82 is undergoing further investigation and experimentation to determine whether it is significantly differentially expressed. AAK13049, as indicated in row 152 and AAK13083 were both identified as proteins indicated by the mass spectral data entered into the Mascot program in FIG. 10.

FIG. 1 through FIG. 10 disclose data analysis that was performed to identify the eleven proteins which are differentially expressed in asthma and/or lung cancer populations when compared to the normal populations. The process described herein, and as indicated in FIG. 1 through FIG. 10 was performed for each of the eleven proteins, for the asthma population, normal population and lung cancer population.

As a result of the identification process, the eleven proteins determined to be significantly differentially expressed between the asthma population, lung cancer population and/or normal population were identified as BAC04615, Q6NSC8, CAF17350, Q6ZUD4, Q8N7P1, CAC69571, FERM domain containing protein 4, JCC1445 proteasome endopeptidase complex chain C2 long splice form, Syntaxin 11, AAK13083, and AAK130490. BAC04615, Q6NSC8, CAF 17350, Q6ZUD4, Q8N7P1 are identified proteins resulting from genetic sequencing efforts. FERM domain containing protein 4 is known to be involved in intracytoplasmic protein membrane anchorage. JCC1445 proteasome endopeptidase complex chain C2 long splice form is a known proteasome. Syntaxin 11 is active in cellular immune response. BAC04615, AAK13083, and AAK130490 are major histocompatibility complex (“MHC”) associated proteins.

Having identified eleven specific proteins which are consistently differentially expressed in asthma and lung cancer patients, it is possible to diagnose these pathologies early in the progression of the diseases by subjecting the proteins BAC04615, Q6NSC8, CAF17350, Q6ZUD4, Q8N7P1, CAC69571, FERM domain containing protein 4, JCC1445 proteasome endopeptidase complex chain C2 long splice form, Syntaxin 11, AAK13083, and AAK130490 from a patient's serum to the LC-ESIMS, obtaining the mass spectral data, from these proteins, and comparing the mass spectral data to mass spectral data of normal populations. Further analysis can be performed by comparing the mass spectral data to mass spectral data from lung cancer populations and/or asthma populations to verify or nullify the presence of the given pathologies.

The analysis could, of course, be extended to multiple additional techniques whereby specific protein concentrations can be determined, including but not limited to: Radio-immuno Assay, enzyme linked immuno sorbent assay, high pressure liquid chromatography with radiometric, spectrometric detection via absorbance of visible or ultraviolet light, mass spectrometric qualitative and quantitative analysis, western blotting, 1 or 2 dimensional gel electrophoresis with quantitative visualization by means of detection of radioactive probes or nuclei, antibody based detection with absorptive or fluorescent photometry, quantitation by luminescence of any of a number of chemiluminescent reporter systems, enzymatic assays, immunoprecipitation or immuno-capture assays, or any of a number of solid and liquid phase immuno assays.

In addition to determining the existence of lung cancer or asthma early in the development of the disease, the proteins identified herein as indicative of such pathologies could be used and applied in related ways to further the goal of treating lung cancer and/or asthma. For instance, antibodies can be developed to bind to these proteins. The antibodies could be assembled in a biomarker panel wherein any or all of the antibodies are assembled into a single bead based panel or kit for a bead based immunoassay. The proteins could then be subjected to a multiplexed immunoassay using bead based technologies, such as Luminex's xMAP technologies, and quantified. Furthermore, other non-bead based assays could be used to quantify the protein expression levels. By quantifying the protein expression levels, those quantifiable results can be compared to expression levels of normal populations, asthma populations, and/or lung cancer populations to further verify or nullify the presence of lung cancer or asthma in the patient.

The proteins could also be used and applied to the field of pharmacology to evaluate the response of a patient to therapeutic interventions such as drug treatment, radiation/chemotherapy, or surgical treatment. Furthermore, kits to measure individual proteins or a panel of the proteins could be used for routine testing of a patient to monitor health status of a patient who is at greater risk of the pathologies, such as smokers, or those with family histories of the pathologies.

Finally, a Sequence Listing the amino acid sequences for each of the eleven proteins identified herein is filed herewith and is specifically incorporated herein by reference. In the Sequence Listing, the amino acid sequence disclosed in SEQ ID NO: 1 is the primary amino acid sequence known as of the date of filing this application for the protein BAC04615. The amino acid sequence disclosed in SEQ ID NO: 2 is the primary amino acid sequence known as of the date of filing this application for the protein Q6NSC8. The amino acid sequence disclosed in SEQ ID NO: 3 is the primary amino acid sequence known as of the date of filing this application for the protein CAF17350. The amino acid sequence disclosed in SEQ ID NO: 4 is the primary amino acid sequence known as of the date of filing this application for the protein Q6ZUD4. The amino acid sequence disclosed in SEQ ID NO: 5 is the primary amino acid sequence known as of the date of filing this application for the protein FERM domain containing protein 4. The amino acid sequence disclosed in SEQ ID NO: 6 is the primary amino acid sequence known as of the date of filing this application for the protein AAK13083. The amino acid sequence disclosed in SEQ ID NO: 7 is the primary amino acid sequence known as of the date of filing this application for the protein Q8N7P1. The amino acid sequence disclosed in SEQ ID NO: 8 is the primary amino acid sequence known as of the date of filing this application for the protein CAC69571. The amino acid sequence disclosed in SEQ ID NO: 9 is the primary amino acid sequence known as of the date of filing this application for the protein JCC1445 proteasome endopetidase complex chain C2 long splice. The amino acid sequence disclosed in SEQ ID NO: 10 is the primary amino acid sequence known as of the date of filing this application for the protein Syntaxin 11. The amino acid sequence disclosed in SEQ ID NO: 11 is the primary amino acid sequence known as of the date of filing this application for the protein AAK13049.

The amino acid sequences disclosed herein and in the Sequence Listing are the primary amino acid sequences which are known as of the filing date of this application. It is to be understood that modifications could be made to the sequences listed in the Sequence Listing for the proteins in the future. For instance, post translational modifications may be discovered which change with the processing of the listed proteins or may form functional adducts to the proteins at some point in their function within the body. In addition, the Sequence Listing may be altered by splicing differences or the discovery of closely structurally related proteins of the same family as the named proteins. Furthermore, proteolytic fragments in all of their permutations arising from the processing or degradation of the listed proteins could produce marker fragments usable in all of the ways that the parent proteins could be exploited in the fields of medicine and pharmacology. Such modifications are contemplated as being within the scope of the invention disclosed herein without departing from the scope of the invention disclosed herein.

Although the invention has been described with reference to specific embodiments, this description is not meant to be construed in a limited sense. Various modifications of the disclosed embodiments, as well as alternative embodiments of the invention will become apparent to persons skilled in the art upon the reference to the description of the invention. It is, therefore, contemplated that the appended claims will cover such modifications that fall within the scope of the invention.

1-21. (canceled)

22. A method of quantifying protein expression levels in a human serum specimen comprising:
obtaining a patient serum specimen;
exposing the patient serum specimen to a digesting agent,
wherein the digesting agent cleaves proteins in the patient serum specimen into defined peptides;
separating the peptides from the patient serum specimen;
subjecting the peptides from the patient serum specimen to analysis using a liquid chromatography mass spectrometer,
wherein the mass spectrometer has a column of hydrophobic stationary phase therein with a solvent system flowing through the column,
wherein the solvent system separates the peptides, and
a detecting mechanism to produce mass spectral readouts,
wherein the mass spectral readouts comprising masses of the peptides and graphic illustrations measuring the intensities of the peptides over time periods that the peptides pass through the column;
selecting at least one of the peptides from the human serum specimen to compare the mass spectral readouts,
wherein the mass spectral readouts of the peptides representing mass spectral readouts of the proteins from which the peptides were cleaved during the exposing step,
wherein the proteins comprise at least one protein selected from the group consisting of CAC69571, FERM domain containing protein 4, JC1445 proteasome endopetidase complex chain C2 long splice, Syntaxin 11, AAK13083, AAK130490, BAC04615, Q6NSC8, CAF17350, Q6ZVD4, Q8N7P1, and combinations thereof;
obtaining mass spectral readouts of intensities of substantially unaltered expressions for each of the same proteins represented from the peptides selected during the selecting step, the intensities of unaltered expressions being determined from a population of human serum specimens not having non-small cell lung cancer;
comparing the mass spectral readouts of the at least one peptide selected during the selecting step from the patient serum specimen to the mass spectral readouts of the unaltered protein expressions from the population of human serum specimens not having non-small cell lung cancer; and
determining whether the intensities of the protein expressions of the patient serum specimen are altered.

23. The method of claim 22, wherein the digesting agent is trypsin or other endoproteinase.

24-45. (canceled)

46. The method of claim 22, wherein the protein expression levels are quantified by determining protein concentrations using a radio-immuno assay, enzyme linked immunosorbent assay, high pressure liquid chromatography with radiometric detection, spectrometric detection using absorbance of visible or ultraviolet light, mass spectrometric qualitative and quantitative analysis, western blotting, 1 or 2 dimensional gel electrophoresis with quantitative visualization using radioactive probes or nuclei, antibody based detection with absorptive or fluorescent photometry, quantitation by luminescence, enzymatic assay, immunoprecipitation or immuno-capture assay, or any solid or liquid phase immunoassay.

47. A method of detecting pathologies of human lung tissues in a patient by quantifying protein expression levels in a human serum specimen of said patient, said method comprising:
quantifying protein expression levels for a protein selected from the group consisting of CAC69571, FERM domain containing protein 4, JC1445 proteasome endopeptidase complex chain C2 long splice, Syntaxin 11, AAK13083, AAK130490, BAC04615, Q6NSC8, CAF17350, Q6ZVD4, Q8N7P1, and combinations thereof, in a human serum specimen from a patient; and
comparing said expression levels to expression levels of corresponding proteins in normal populations and/or lung cancer populations,
wherein differential expression levels of one or more of said proteins is indicative of non-small cell lung cancer.

48. The method of claim 47, wherein the protein expression levels are quantified by determining protein concentrations using a radio-immuno assay, enzyme linked immunosorbent assay, high pressure liquid chromatography with radiometric detection, spectrometric detection using absorbance of visible or ultraviolet light, mass spectrometric qualitative and quantitative analysis, western blotting, 1 or 2 dimensional gel electrophoresis with quantitative visualization using radioactive probes or nuclei, antibody based detection with absorptive or fluorescent photometry, quantitation by luminescence, enzymatic assay, immunoprecipitation or immuno-capture assay, or any solid or liquid phase immunoassay.


SEQUENCE LISTING (11 sequences)

SEQ ID NO: 1 - protein, 319 aa, Homo sapien
MVLSELAARLNCAEYKNWVKAGHCLLLLRSCLQGFVGREVLSFHRGLLAAAPGLGPRAVCRGGSRCSPRARQFQPQCQVCAEWKREILRHHVNRNGDVHWGNCRPGRWPVDAWEVAKAFMPRGLADKQGPEECDAVALLSLINSCDHFVVDRKKVTEVIKCRNEIMHSSEMKVSSTWLRDFQMKIQNFLNEFKNIPEIVAVYSRIEQLLTSDWAVHIPEEDQRDGCECEMGTYLSESQVNEIEMQLLKEKLQEIYLQAEEQEVLPEELSNRLEVVKEFLRNNEDLRNGLTEDMQKLDSLCLHQKLDSQEPGRQTPDRKA

SEQ ID NO: 2 - protein, 57 aa, Homo sapiens [sequence omitted]

SEQ ID NO: 3 - protein, 62 aa, Homo sapiens
MIRSKFRVPRILHVLSAHSQASDKNFTAENSEVVVSSRTDVSPMKSDLLLPPSKPGCNNVLN

SEQ ID NO: 4 - protein, 146 aa, Homo sapiens
MVQGMCSPSPFGTSRACTVGTQVDSRSLPWALGASAQRGNIPTATCARTAGTLRRGLQPGWGWEDFLDEGQPGFSSRMSWSRPPAQEQGAGRGPSWVRGLGQPTAAFEQGPRSSVSPQWEGGGQGPGELGRKHLLGPSQHHPTDRH

SEQ ID NO: 5 - protein, 1039 aa, Homo sapiens
MAVQLVPDSALGLLMMTEGRRCQVHLLDDRKLELLVQPKLLAKELLDLVASHFNLKEKEYFGIAFTDETGHLNWLQLDRRVLEHDFPKKSGPVVLYFCVRFYIESISYLKDNATIELFFLNAKSCIYKELIDVDSEVVFELASYILQEAKGDFSSNEVVRSDLKKLPALPTQALKEHPSLAYCEDRVIEHYKKLNGQTRGQAIVNYMSIVESLPTYGVHYYAVKDKQGIPWWLGLSYKGIFQYDYHDKVKPRKIFQWRQLENLYFREKKFSVEVHDPRRASVTRRTFGHSGIAVHTWYACPALIKSIWAMAISQHQFYLDRKQSKSKIHAARSLSEIAIDLTETGTLKTSKLANMGSKGKIISGSSGSLLSSGSQESDSSQSAKKDMLAALKSRQEALEETLRQRLEELKKLCLREAELTGKLPVEYPLDPGEEPPIVRRRIGTAFKLDEQKILPKGEEAELERLEREFAIQSQITEAARRLASDPNVSKKLKKQRKTSYLNALKKLQEIENAINENRIKSGKKPTQRASLIIDDGNIASEDSSLSDALVLEDEDSQVTSTISPLHSPHKGLPPRPPSHNRPPPPQSLEGLRQMHYHRNDYDKSPIKPKMWSESSLDEPYEKVKKRSSHSHSSSHKRFPSTGSCAEAGGGSNSLQNSPIRGLPHWNSQSSMPSTPDLRVRSPHYVHSTRSVDISPTRLHSLALHFRHRSSSLESQGKLLGSENDTGSPDFYTPRTRSSNGSDPMDDCSSCTSHSSSEHYYPAQMNANYSTLAEDSPSKARQRQRQRQRAAGALGSASSGSMPNLAARGGAGGAGGAGGGVYLHSQSQPSSQYRIKEYPLYIEGGATPVVVRSLESDQEGHYSVKAQFKTSNSYTAGGLFKESWRGGGGDEGDTGRLTPSRSQILRTPSLGREGAHDKGAGRAAVSDELRQWYQRSTASHKEHSRLSHTSSTSSDSGSQYSTSSQSTFVAHSRVTRMPQMCKATSAALPQSQRSSTPSSEIGATPPSSPHHILTWQTGEATENSPILDGSESPPHQSTDE

SEQ ID NO: 6 - protein, 244 aa, Homo sapiens
MAAAASPAILPRLAILPYLLFDWSGTGRADAHSLWYNFTIIHLPRHGQQWCEVQSQVDQKNFLSYDCGSDKVLSMGHLEEQLYATDAWGKQLEMLREVGQRLRLELADTELEDFTPSGPLTLQVRMSCECEADGYIRGSWQFSFDGRKFLLFDSNNRKWTVVHAGARRMKEKWEKDSGLTTFFKMVSMRDCKSWLRDFLMHRKKRLEPTAPPTMAPGLAQPKAIATTLSPWSFLIILCFILPGI

SEQ ID NO: 7 - protein, 536 aa, Homo sapiens
MEIRQHEWLSASPHEGFEQMRLKSRPKEPSPSLTRVGANFYSSVKQQDYSASVWLRRKDKLEHSQQKCIVIFALVCCFAILVALIFSAVDIMGEDEDGLSEKNCQNKCRIALVENIPEGLNYSENAPFHLSLFQGWMNLLNMAKKSVDIVSSHWDLNHTHPSACQGQRLFEKLLQLTSQNIEIKLVSDVTADSKVLEALKLKGAEVTYMNMTAYNKGRLQSSFWIVDKQHVYIGSAGLDWQSLGQMKELGVIFYNCSCLVLDLQRIFALYSSLKFKSRVPQTWSKRLYGVYDNEKKLQLQLNETKSQAFVSNSPKLFCPKNRSFDIDAIYSVIDDAKQYVYIAVMDYLPISSTSTKRTYWPDLDAKIREALVLRSVRVRLLLSFWKETDPLTFNFISSLKAICTEIANCSLKVKFFDLERENACATKEQKNHTFPRLNRNKYMVTDGAAYIGNFDWVGNDFTQNAGTGLVINQADVRNNRSIIKQLKDVFERDWYSPYAKTLQPTKQPNCSSLFKLKPLSNKTATDDTGGKDPRNV

SEQ ID NO: 8 - protein, 344 aa, Homo sapiens
QNLPSSPAPSTIFSGGFRHGSLISIDSTCTEMGNFDNANVTGEIEFAIHYCFKTHSLEICIKACKNLAYGEEKKKKCNPYVKTYLLPDRSSQGKRKTGVQRNTVDPTFQETLKYQVAPAQLVTRQLQVSVWHLGTLARRVFLGEVIISLATWDFEDSTTQSFRWHPLRAKAEKYEDSVPQSNGELTVRAKLVLPSRPRKLQEAQEGTDQPSLHGQLCLVVLGAKNLPVRPDGTLNSFVKGCLTLPDQQKLRLKSPVLRKQACPQWKHSFVFSGVTPAQLRQSSLELTVWDQALFGMNDRLLGGTRLGSKGDTAVGGDACSLSKLQWQKVLSSPNLWTDMTLVLH

SEQ ID NO: 9 - protein, 263 aa, Homo sapiens
MFRNQYDNDVTVWSPQGRIHQIEYAMEAVKQGSATVGLKSKTHAVLVALKRAQSELAAHQKKILHVDNHIGISIAGLTADARLLCNFMRQECLDSRFVFDRPLPVSRLVSLIGSKTQIPTQRYGRRPYGVGLLIAGYDDMGPHIFQTCPSANYFDCRAMSIGARSQSARTYLERHMSEFMECNLNELVKHGLRALRETLPAEQDLTTKNVSIGIVGKDLEFTIYDDDDVSPFLEGLEERPQRKAQPAQPADEPAEKADEPMEH

SEQ ID NO: 10 - protein, 287 aa, Homo sapiens
MKDRLAELLDLSKQYDQQFPDGDDEFDSPHEDIVFETDHILESLYRDIRDIQDENQLLVADVKRLGKQNARFLTSMRRLSSIKRDTNSIAKAIKARGEVIHCKLRAMKELSEAAEAQHGPHSAVARISRAQYNALTLTFQRAMHDYNQAEMKQRDNCKIRIQRQLEIMGKEVSGDQIEDMFEQGKWDVFSENLLADVKGARAALNEIESRHRELLRLESRIRDVHELFLQMAVLVEKQADTLNVIELNVQKTVDYTGQAKAQVRKAVQYEEKNPCRTLCCFCCPCLK

SEQ ID NO: 11 - protein, 244 aa, Homo sapiens; identical to SEQ ID NO: 6